(19) United States
(12) Patent Application Publication
(10) Pub. No.: US 2025/0128220 A1
(43) Pub. Date: Apr. 24, 2025

(54) SYSTEMS WITH IMPROVED HYDROGEL FORMATION TIMES

(71) Applicant: Boston Scientific Scimed, Inc., Maple Grove, MN (US)

(72) Inventors: Yen-Hao Hsu, Shrewsbury, MA (US); Heather Cirka, Hudson, MA (US); Marc Plourde, Worcester, MA (US); Alexis Compton, Wrentham, MA (US); Anthony Hannan, Cork (IE)

(73) Assignee: Boston Scientific Scimed, Inc., Maple Grove, MN (US)

(21) Appl. No.: 18/918,996

(22) Filed: Oct. 17, 2024

Related U.S. Application Data
(60) Provisional application No. 63/591,681, filed on Oct. 19, 2023.

(51) Int. Cl. B01F 35/71; A61K 9/00; A61K 47/10; A61K 47/18; A61L 27/02; A61L 27/18; A61L 27/52; B01F 21/00; B01F 21/20; B01F 23/40; B01F 23/451; B01F 31/65; B01F 101/00

(52) U.S. Cl. CPC B01F 35/7164; A61K 9/0024; A61K 47/10; A61K 47/18; A61L 27/025; A61L 27/18; A61L 27/52; B01F 21/02; B01F 21/22; B01F 23/405; B01F 23/451; B01F 31/651; B01F 35/712; B01F 35/7131; B01F 35/7174; A61L 2430/34; B01F 2101/2204; B01F 2101/2805; B01F 2215/0431; B01F 2215/044; B01F 2215/0477

(57) ABSTRACT

In some aspects, the present disclosure pertains to systems for forming hydrogel compositions, the systems comprising a first reservoir containing an iodinated polymer composition that comprise an iodinated polymer having multiple cyclic imide ester groups, a second reservoir containing a buffered diluent solution having a first pH that comprises water, a first buffering agent and a polyamine compound, and a third reservoir containing a buffered accelerant solution having a second pH that is higher than the first pH and comprises water and a second buffering agent. Other aspects of the present disclosure pertain to methods of using such systems for forming hydrogel compositions.

### CROSS-REFERENCE TO RELATED APPLICATIONS

This application claims the benefit of U.S. Provisional Patent Application Ser. No. 63/591,681 filed on Oct. 19, 2023, the disclosure of which is incorporated herein by reference.

### FIELD

The present disclosure relates to systems and kits for use in forming hydrogels and to methods of making hydrogels using such systems.

### BACKGROUND

SpaceOAR® is a rapid crosslinking hydrogel that polymerizes in vivo and is based on a multi-arm polyethylene glycol (PEG) polymer functionalized with succinimidyl glutarate as activated end groups which further react with trilysine to form crosslinks. This product has been used clinically in prostate cancer therapy. A further improvement based on this system is that a portion the succinimidyl glutarate end groups are replaced with 2,3,5-triiiodobenzamide groups, providing radiopacity. This hydrogel, known by the trade name of SpaceOAR® Vue, is the radiopaque version of SpaceOAR® for prostate medical applications. During application of SpaceOAR® and SpaceOAR® Vue, two solutions (a) a solution of trilysine and multi-arm-PEG in an acidic phosphate buffer and (b) an accelerant solution comprising a basic buffer containing sodium tetraborate decahydrate are mixed together via a Y-connector and simultaneously injected into a space between the rectum and prostate via an 18-gauge needle. When mixed, the pH surrounding the trilysine and the multi-arm-PEG increases, causing the trilysine and the multi-arm-PEG to crosslink and form a solid hydrogel. This crosslinking reaction is illustrated schematically for SpaceOAR® Vue in FIG. 1, where the multi-arm-PEG is 8-arm PEG, R represents a polyol residue core, and n is an integer of approximately 28. In the case of either SpaceOAR® or SpaceOAR® Vue, the hydrogel persists for approximately 6 months and then gradually dissipates via hydrolysis. The gelation time of the SpaceOAR® as measured by a bench-top test method is about 5 to 7 seconds, whereas the gelation time of the SpaceOAR® Vue is about 11 seconds or longer. This increased gel time potentially results in less precise hydrogel placement.

It would be desirable to have a radiopaque hydrogel-forming system that has shorter gel time for reliable product placement.

### SUMMARY

In some aspects, the present disclosure pertains to a system for forming a hydrogel composition that comprises a first reservoir containing an iodinated polymer composition that comprise an iodinated polymer having multiple cyclic imide ester groups, a second reservoir containing a buffered diluent solution having a pH ranging from 3.8-4.2 that comprises water, a first buffering agent and a polyamine compound, and a third reservoir containing a buffered accelerant solution having a pH ranging from 10.8-11.2 that comprises water and a second buffering agent.

In other aspects, the present disclosure pertains to a system for forming a hydrogel composition that comprises a first reservoir containing an iodinated polymer composition that comprise an iodinated polymer having multiple cyclic imide ester groups, a second reservoir containing a buffered diluent solution having a pH ranging from 4.4-5.5 that comprises water, a first buffering agent and a polyamine compound, and a third reservoir containing a buffered accelerant solution having a pH ranging from 10.2-10.6 that comprises water and a second buffering agent.

In some embodiments, when using systems in accordance with any of the above aspects, upon dissolving the iodinated polymer composition in the buffered diluent solution to form a buffered precursor solution containing water, the first buffering agent, the iodinated polymer and the polyamine, and upon mixing the buffered precursor solution with the buffered accelerant, crosslinking between the iodinated polymer and the polyamine leads to hydrogel formation. For example, a gel formation time for hydrogel formation may range from 5 to 8 seconds when the buffered precursor solution is combined with the buffered accelerant solution within 10 minutes of forming the precursor solution.

In some embodiments, which can be used in conjunction with any of the above aspects and embodiments, the first buffering agent comprises a phosphate buffering agent.

In some embodiments, which can be used in conjunction with any of the above aspects and embodiments, the first buffering agent comprises sodium phosphate monobasic.

In some embodiments, which can be used in conjunction with any of the above aspects and embodiments, the second buffering agent comprises a borate buffering agent and a phosphate buffering agent.

In some embodiments, which can be used in conjunction with any of the above aspects and embodiments, the second buffering agent comprises sodium tetraborate decahydrate and dibasic sodium phosphate.

In some embodiments, which can be used in conjunction with any of the above aspects and embodiments, one, two or all three of the iodinated polymer composition, the buffered diluent solution or the buffered accelerant solution may further comprise one or more additional agents selected from therapeutic agents, imaging agents, colorants, tonicity adjusting agents, suspension agents, wetting agents, and pH adjusting agents.

In some embodiments, which can be used in conjunction with any of the above aspects and embodiments, the first, second and third reservoirs may independently be selected from vials and syringes.

In some embodiments, which can be used in conjunction with any of the above aspects and embodiments, the first reservoir is a vial, the second reservoir is a first syringe comprising a first syringe barrel terminating in a connector and a first plunger, and the third reservoir is a second syringe comprising a second syringe barrel terminating in a connector and a second plunger.

In some of these embodiments, the systems further comprises (a) a needle or a tube and (b) a Y-connector that comprises a first branch lumen having a first end and a second end, a second branch lumen having a first end and a second end, and a common lumen having a first end and a second end, the first end of the first branch lumen and the first end of the second branch lumen in fluid communication with the first end of the common lumen, the second end of the first branch lumen terminating at a connector which is configured to connect with the connector of the first syringe barrel, the second end of the second branch lumen terminating at a connector which is configured to connect with the connector of the second syringe barrel, and the second end of the common lumen terminating at a connector configured to connect to a connector of the needle or the tube. For example, the first branch lumen and the second branch lumen may have an internal diameter ranging from 12 gauge to 19 gauge.

In some of the above embodiments, the system further comprises one, two of all three of the following: a syringe holder that is configured to hold the first and second syringe barrels in a fixed relationship, a plunger cap that is configured to hold the first and second plungers in a fixed relationship, and a vial adapter configured for providing fluid communication between the syringe barrel and the vial.

In other aspects, the present disclosure pertains to kits that comprise a sterile package comprising components of a system in accordance with any of the above aspects and embodiments in one or more trays.

In other aspects the present disclosure pertains to a methods of using such systems and kits, which methods comprise: injecting the buffered diluent solution from the first syringe into the vial containing the iodinated polymer composition, thereby forming a buffered precursor solution containing water, the first buffering agent, the iodinated polymer and the polyamine; pulling the buffered precursor solution back into the first syringe; connecting the first syringe barrel of the first syringe to the first branch lumen of the Y-connector; connecting the second syringe barrel of the second syringe to the second branch lumen of the Y-connector; connecting the needle or tube to the common lumen of the Y-connector, and simultaneously pressing the first plunger and the second plunger into the first syringe barrel and the second syringe barrel, respectively.

In some embodiments, the method further comprises: attaching the syringe holder to the first and second syringe barrels, thereby holding the first and second syringe barrels in a fixed relationship, and attaching the plunger cap to the first and second plungers, thereby holding the first and second plungers in a fixed relationship.

The above and other aspects, embodiments and features of the present disclosure will be readily apparent from the following detailed description.

### BRIEF DESCRIPTION OF THE DRAWINGS

FIG. 1 schematically illustrates a chemical crosslinking process, in accordance with an embodiment of the present disclosure.

FIG. 2 schematically illustrates an injection apparatus, in accordance with an embodiment of the present disclosure.

FIG. 3 schematically illustrates a Y-connector, in accordance with an embodiment of the present disclosure.

FIGS. 4A-4B schematically illustrate measured injection force, in accordance with embodiments of the present disclosure.

FIG. 5 schematically illustrates measured modulus, in accordance with an embodiment of the present disclosure.

FIG. 6 schematically illustrates measured gel persistence, in accordance with an embodiment of the present disclosure.

FIG. 7 schematically illustrates measured percent swell, in accordance with an embodiment of the present disclosure.

FIGS. 8A-8B schematically illustrate measured gel times, in accordance with embodiments of the present disclosure.

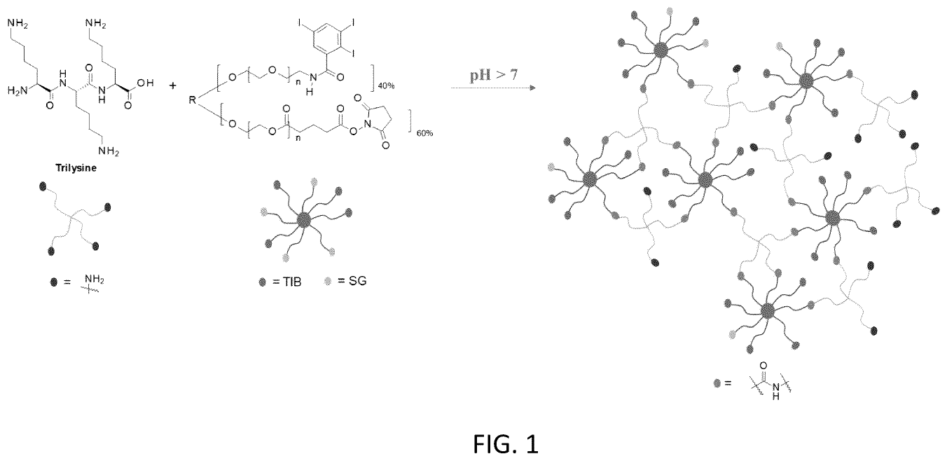

### DETAILED DESCRIPTION

In some aspects, the present disclosure provides a system for forming a hydrogel composition that comprises a first reservoir containing an iodinated polymer composition that comprise an iodinated polymer having multiple cyclic imide ester groups, a second reservoir containing a buffered diluent solution having a pH ranging from 3.8 to 4.2, more typically from 3.9-4.1, that comprises water, a first buffering agent and a polyamine compound, and a third reservoir containing a buffered accelerant solution having a pH ranging from 10.8-11.2, more typically from 10.9-11.1, that comprises water and a second buffering agent.

In some aspects, the present disclosure provides a system for forming a hydrogel composition that comprises a first reservoir containing an iodinated polymer composition that comprise an iodinated polymer having multiple cyclic imide ester groups, a second reservoir containing a buffered diluent solution having a pH ranging from 4.4-5.5, more typically from 4.4 to 4.8, even more typically from 4.5 to 4.7, that comprises water, a first buffering agent and a polyamine compound, and a third reservoir containing a buffered accelerant solution having a pH ranging from 10.2-10.6, more typically from 10.3 to 10.5, that comprises water and a second buffering agent.

During use, the iodinated polymer composition in the first reservoir may be dissolved in the buffered diluent solution to form a buffered precursor solution containing water, the first buffering agent, the iodinated polymer and the polyamine. The buffered precursor solution is then combined with the buffered accelerant solution in the third reservoir, which increases the pH of the resulting mixture, causing accelerated crosslinking between the iodinated polymer and the polyamine, which leads to the formation of a hydrogel. Gel formation times may range, for example, from 5 to 8 seconds when the buffered precursor solution is combined with the buffered accelerant solution within 10 minutes of forming the precursor solution.

The first, second and third reservoirs may independently be selected from, for example, vials and syringes. In particular embodiments, the first reservoir may be a vial, which may contain the iodinated polymer composition in dry form (e.g., as a powder), the second reservoir may be a first syringe comprising a first syringe barrel terminating in a connector (e.g., a Luer connector) and a first plunger, and the third reservoir may be a second syringe comprising a second syringe barrel terminating in a connector (e.g., a Luer connector) and a second plunger.

The system may also include a Y-connector that is configured to combine and mix the contents of the first and second syringes into a combined stream which can then be injected into the patient, for example, through a needle or a tube. For example, the Y-connector may include a first branch lumen having a first end and a second end, a second branch lumen having a first end and a second end, and a common lumen having a first end and a second end. The first end of the first branch lumen and the first end of the second branch lumen may be in fluid communication with the first end of the common lumen at a merge point. The second end of the first branch lumen may terminate at a connector (e.g., a Luer connector) which is configured to connect with a complementary connector of the first syringe barrel, the second end of the second branch lumen may terminate at a connector (e.g., a Luer connector) which is configured to connect with a complementary connector of the second syringe barrel, and the second end of the common lumen may terminate at a connector (e.g., a Luer connector) which is configured to connect with a complementary connector of a needle or a tube (e.g., a Luer connector).

The system may optionally further include one, two or all three of the following: a syringe holder that is configured to hold the first and second syringe barrels in a fixed relationship, a plunger cap that is configured to hold the first and second plungers in a fixed relationship, and a vial adapter for providing fluid communication between the syringe barrel and the vial. Such a vial adaptor may include a spike, which is configured for the puncturing an elastomeric closure of the vial containing the iodinated polymer composition, thereby accessing the interior of the vial, and a connector (e.g., Luer connector), which is configured for attachment to the first syringe barrel.

During use, the buffered diluent solution may be injected from the first syringe into to the vial containing the iodinated polymer composition (e.g., using the vial adaptor), resulting in a buffered precursor solution containing water, the first buffering agent, the iodinated polymer and the polyamine, after which the buffered precursor solution is pulled back into the first syringe. The vial may be shaken prior to retraction of the buffered precursor solution into the first syringe to ensure that the iodinated polymer composition is dissolved.

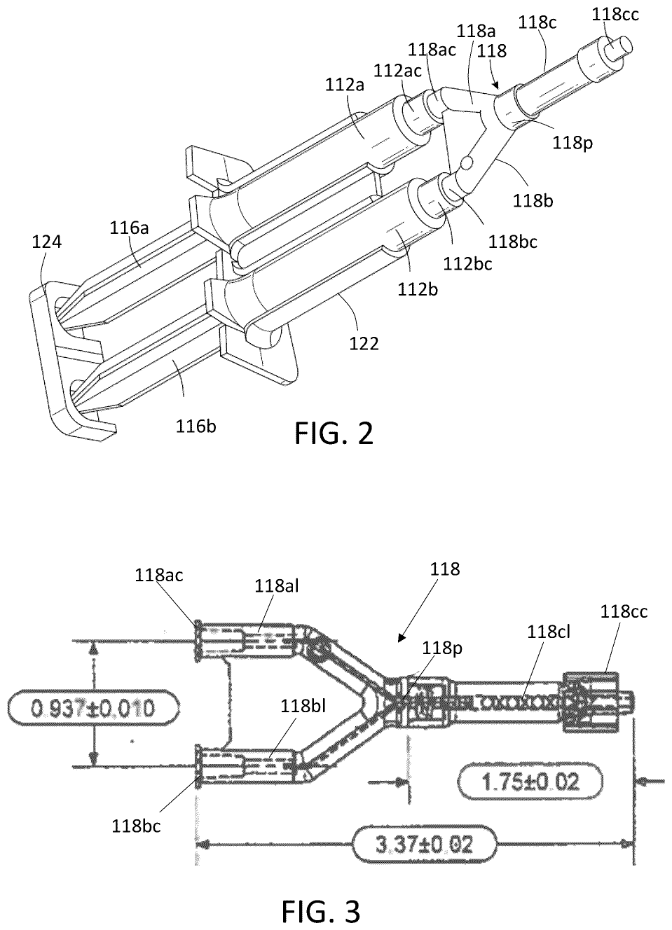

With reference now to FIG. 2, a connector 112ac of the first syringe barrel 112a containing the buffered precursor solution is connected to a connector 118ac of a first branch 118a of the Y-connector, and a connector 112bc of the second syringe barrel 112b containing the buffered accelerant solution is connected to a connector 118bc of a second branch 118b of the Y-connector. The Y-connector further includes common branch 118c having a common branch connector 118cc. A region 118p is shown, where the lumens of the first branch 118a and the second branch 118b merge into the lumen of the common branch 118c. Also shown are a first plunger 116a that is movable in the first syringe barrel 112a, a second plunger 116b that is movable in the second barrel 112b, a syringe holder 122 configured to hold the first and second syringe barrels 112a, 112b, in a fixed relationship and a plunger cap 124 configured to hold the first and second plungers 116a, 116b in a fixed relationship. After the first syringe barrels 112a, 112b are attached to the Y-connector, the syringe holder 122 is attached to the first and second syringe barrels 112a, 112b, and the plunger cap 124 is attached to the first and second plungers 116a, 116b. A needle or tube (not shown) is attached to the common branch connector 118cc.

During operation, the first plunger 116a is pressed into the first syringe barrel 112a, forcing the buffered precursor solution through the lumen of the first branch 118a of the Y-connector. Simultaneously, the second plunger 116b is pressed into the second syringe barrel 112b, forcing the buffered accelerant solution through lumen of the second branch 118b of the Y-connector. The buffered precursor solution and the buffered accelerant solution meet and mix at the merge point 118p, with the resultant mixture moving along the lumen of the common branch 118c and exiting the Y-connector 118 at the common branch connector 118cc. As previously noted, combining the buffered accelerant solution with the buffered precursor solution increases the pH of the resulting mixture, causing crosslinking between the iodinated polymer and the polyamine, which leads to the formation of a hydrogel.

A particular embodiment of a Y-connector 118 having an overall length of 3.37±0.02 inches is shown in FIG. 3 and includes a first branch lumen 118al terminating at a Luer connector 118ac, a second branch lumen 118bl terminating at a Luer connector 118bc and a common lumen 118cl terminating at a Luer connector 118ac. The distance between Luer connector 118ac and Luer connector 118bc is 0.937±0.010 inches, the length of the common lumen 118cl is 1.75±0.02 inches, and the overall length of the Y-connector is 3.37±0.02 inches. The first branch lumen 118al and the second branch lumen 118bl are formed using differing hypotubes having inside diameters that are described below.

In some embodiments of the present disclosure, kits are provided that include a first reservoir (e.g., a vial or syringe) containing an iodinated polymer composition as described herein, a second reservoir (e.g., a vial or syringe) containing a buffered diluent solution as described herein, a third reservoir (e.g., a vial or syringe) containing a buffered accelerant solution as described herein, additional apparatus, as required, for combining the iodinated polymer composition and buffered diluent solution to provide a buffered precursor solution, and additional apparatus for combining and delivering the buffered precursor solution and buffered accelerant solution to a patient.

In particular embodiments, the kits may comprise a vial containing an iodinated polymer composition as described herein, a first syringe containing a buffered diluent solution as described herein, a second syringe containing a buffered accelerant solution as described herein, a needle and/or tube, a Y-connector, a syringe holder, a plunger cap and a vial adapter. Such components may be placed in sterile packaging, for example, in one or more packaged sterile trays.

Iodinated polymers having multiple cyclic imide ester groups for use herein include iodinated polymers that comprise a plurality of polymer arms. A first portion of the polymer arms (for example, from 50 to 70 mol % of the polymer arms, beneficially, from 55 to 65 mol % of the polymer arms, more beneficially, about 60 mol % of the polymer arms) of the iodinated multi-arm polymer may comprise a hydrophilic polymer segment and a reactive cyclic imide ester end group and a second portion of the polymer arms (for example, from 30 to 50 mol % of the polymer arms, beneficially, from 35 to 45 mol % of the polymer arms, more beneficially, about 40 mol % of the polymer arms) of the iodinated multi-arm polymer may comprise a hydrophilic polymer segment and an iodinated aromatic end group.

Iodinated multi-arm polymers in accordance with the present disclosure include iodinated multi-arm polymers having from 4 to 20 arms, beneficially from 6 to 10 arms, more beneficially 8 arms.

Examples of cyclic imide ester groups, include succinimidyl ester groups, maleimidyl ester groups, glutarimidyl ester groups, phthalimidyl ester groups, and bicyclo[2.2.1]hept-5-ene-2,3-dicarboxylic acid imidyl ester groups, among others.

Examples of iodinated aromatic groups include iodobenzamide groups including monoiodobenzamide groups (e.g., 2-iodobenzamide groups, 3-iodobenzamide groups, 4-iodobenzamide groups), diiodobenzamide groups (e.g., 2,5-diiodobenzamide groups, 3,5-diiodobenzamide groups, 3,4-diiodobenzamide groups, etc.) and triiodobenzamide groups (e.g., 2,3,4-triiodobenzamide groups, 2,4,6-triiodobenzamide groups, etc.) and hydoxyiodobenzamide groups including hydroxymonoiodobenzamide groups (e.g., 4-hydroxy-3-iodobenzamide groups, 2-hydroxy-4-iodobenzamide groups, 3-hydroxy-4-iodobenzamide groups, etc.), hydroxydiiodobenzamide groups (e.g., 2-hydroxy-3,5-diiodobenzamide groups, 4-hydoxy-3,5-diiodobenzamide groups, etc.), and hydroxytriiodobenzamide groups (e.g., 3-hydroxy-2,4,6-triiodobenzamide groups, etc.).

The cyclic imide ester and iodinated aromatic groups may be linked to the hydrophilic polymer segment through any suitable linking group, which may be selected, for example, from a linking group that comprises an alkyl group, a linking group that comprises an ether group, a linking group that comprises an ester group, a linking group that comprises an amide group, a linking group that comprises an amine group, a linking group that comprises a carbonate group, or a linking group that comprises a combination of two or more of the foregoing groups, among others.

In certain embodiments, the cyclic imide ester may be linked to the hydrophilic polymer segment through a linking group that comprises a hydrolysable ester group. Examples include, for example, succinimidyl malonate groups, succinimidyl glutarate groups, succinimidyl succinate groups, succinimidyl adipate groups, succinimidyl diglycolate groups, maleimidyl malonate groups, maleimidyl glutarate groups, maleimidyl succinate groups, maleimidyl adipate groups, maleimidyl diglycolate groups, glutarimidyl malonate groups, glutarimidyl glutarate groups, glutarimidyl succinate groups, glutarimidyl adipate groups, glutarimidyl diglycolate groups, phthalimidyl malonate groups, phthalimidyl glutarate groups, phthalimidyl succinate groups, phthalimidyl adipate groups, phthalimidyl diglycolate groups, bicyclo[2.2.1]hept-5-ene-2,3-dicarboxylic acid imidyl malonate groups, bicyclo[2.2.1]hept-5-ene-2,3-dicarboxylic acid imidyl glutarate groups, bicyclo[2.2.1]hept-5-ene-2,3-dicarboxylic acid imidyl succinate groups, bicyclo[2.2.1]hept-5-ene-2,3-dicarboxylic acid imidyl adipate groups, and bicyclo[2.2.1]hept-5-ene-2,3-dicarboxylic acid imidyl diglycolate groups, among others.

Hydrophilic polymer segments may be selected, for example, from the following polymer segments: polyether segments including poly(C₁-C₆-alkylene oxide) segments such as poly(ethylene oxide) (PEO) segments (also referred to as polyethylene glycol or PEG segments), poly(propylene oxide) segments, poly(ethylene oxide-co-propylene oxide) segments, polymer segments formed from one or more polar aprotic vinyl monomers, including poly(N-vinyl pyrrolidone) segments, poly(acrylamide) segments, poly(N-methyl acrylamide) segments, poly(dimethyl acrylamide) segments, poly(N-vinylimidazole) segments, poly(4-vinylimidazole) segments, and poly(sodium 4-vinylbenzenesulfonate) segments, polydioxanone segments, polyester segments including polyglycolide segments, polylactide segments, poly(lactide-co-glycolide) segments, poly(β-propiolactone) segments, poly(β-butyrolactone) segments, poly(γ-butyrolactone) segments, poly(γ-valerolactone) segments, poly(δ-valerolactone) segments, and poly(ε-caprolactone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, poly(N-isopropylacrylamide) segments, polypeptide segments, and polysaccharide segments.

Polymer segments for use in the multi-arm polymers of the present disclosure typically contain between 10 and 1000 monomer units.

In various embodiments, the polymer arms of the iodinated multi-arm polymer may extend from a core region that comprises a residue of a polyol comprising three or more hydroxyl groups, which is used to form the polymer arms. In certain beneficial embodiments, the core region comprises a residue of a polyol that contains from 3 to 20 hydroxyl groups, beneficially from 6 to 10 hydroxyl groups, more beneficially 8 hydroxyl groups.

Illustrative polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols, such as glycerol, mannitol, sorbitol, inositol, xylitol, quebrachitol, threitol, arabitol, erythritol, pentaerythritol, dipentaerythritol, tripentaerythritol, adonitol, hexaglycerol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl)ethane, and 2,6-bis(hydroxyalkyl)cresols, among others.

Polyamines for use in the buffered diluent solutions of the present disclosure include polyamines that comprise basic amino acid residues, including residues of amino acids having two or more primary amine groups, such as lysine and ornithine, for example, polyamines that comprise from 2 to 10 lysine and/or ornithine amino acid residues (e.g., dilysine, trilysine, tetralysine, pentalysine, diornithine, triornithine, tetraornithine, pentaornithine, etc.). Polyamine compounds which may be used as the polyamine compound further include ethylenetriamine, diethylene triamine, hexamethylenetriamine, di(heptamethylene) triamine, di(trimethylene) triamine, bis(hexamethylene) triamine, triethylene tetramine, tripropylene tetramine, tetraethylene pentamine, hexamethylene heptamine, pentaethylene hexamine, dimethyl octylamine, dimethyl decylamine, and JEFFAMINE polyetheramines available from Huntsman Corporation, chitosan and derivatives thereof, poly(vinyl amine), and poly(allyl amine), among others.

First buffering agents for use in the buffered diluent solutions of the present disclosure include those that are capable of providing solutions having a pH ranging from 3.8 to 6.5, more typically from 3.8 to 5.5. Particular examples of such first buffering agents include phosphate buffering agents, such as sodium phosphate monobasic.

Second buffering agents for use in the buffered accelerant solutions of the present disclosure include those that are capable of providing solutions having a pH ranging from 10.2 to 12.0, more typically from 10.2 to 11.2. Particular examples of such second buffering agents include a combination of borate and phosphate buffering agents (e.g., sodium tetraborate decahydrate and dibasic sodium phosphate) and NaOH.

Additional agents may be added to any of the iodinated polymer composition, the buffered diluent solution and the buffered accelerant solution described hereinabove. Examples of additional agents include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, matrix metalloproteinase inhibitors, Bcl-2 inhibitors, DNA alkylating agents, spindle poisons, poly (DP-ribose) polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (e) imageable radioisotopes including 99mTc, 201Th, 51Cr, 67Ga, 68Ga, 111 In, 64Cu, 89Zr, 59Fe, 42K, 82Rb, 24Na, 45Ti, 44Sc, 51Cr and 177Lu, among others, and (f) additional radiocontrast agents, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block polymers, etc.), among others, and pH adjusting agents including various buffer solutes.

The systems described herein may be used for in a variety of medical procedures.

For example, the systems and kits of the present disclosure may be used to provide fiducial markers, to provide tissue augmentation or regeneration, to provide a filler or replacement for soft tissue, to provide mechanical support for compromised tissue, to provide a scaffold, as a carrier of therapeutic agents in the treatment of diseases and cancers and the repair and regeneration of tissue, among other uses.

The systems and kits of the present disclosure may be used in a variety of medical procedures, including the following, among others: a procedure to implant a fiducial marker comprising a hydrogel, a procedure to implant a tissue regeneration scaffold comprising a hydrogel, a procedure to implant a tissue support comprising a hydrogel, a procedure to implant a tissue bulking agent comprising a hydrogel, a procedure to implant a therapeutic-agent-releasing depot comprising a hydrogel, a tissue augmentation procedure comprising implanting a hydrogel, a procedure to introduce a hydrogel between a first tissue and a second tissue to space the first tissue from the second tissue.

The systems and kits of the present disclosure may be used in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

### Example 1

To determine the effect of modifying buffered accelerant solution pH on gel properties, five tests were completed, specifically, gel persistence, % swell, gel modulus, maximum injection force, and gel time.

To provide a modified buffered accelerant solution, a stir bar was placed into a 100 mL beaker, and accelerator syringes from existing SpaceOAR® Vue kits were expelled into the 100 mL beaker. The buffered accelerant solution contained a combination of sodium tetraborate decahydrate and dibasic sodium phosphate as buffering agents. Once all accelerator syringes from the kits were emptied into the beaker (15 syringes were emptied to test injection force and gel time and 5 syringes were emptied to test modulus and persistence), the initial pH of the combined solution was measured. NaOH was then added to the combined accelerant solution to increase the pH of the solution. In particular, NaOH was titrated until the pH was approximately 11. For the experiments with 15 syringes approximately 430 μL of NaOH was added (VWR Chemicals, 6.0N). For the experiments with 5 syringes about 200 μL of NaOH was added. When the desired pH was reached, the respective accelerator syringes were refilled by pulling 5 mL of the modified accelerant solution back into the syringes. The existing kits also included a vial containing 8-arm PEG powder in which approximately 60% of the polymer arms have succinimidyl glutarate end groups and approximately 40% of the polymer arms have 2,3,5-triiiodobenzamide end groups, a diluent syringe containing a buffered diluent solution of trilysine and sodium phosphate monobasic (pH=3.8-4.0), a Y-connector, an 18 G×15 cm needle, a syringe holder, a plunger cap and a vial adapter.

Using the vial adaptor, buffered diluent solution was introduced into the vial to dissolve the PEG powder and form a precursor solution, which was drawn back into the syringe. The syringe containing the precursor solution, the syringe containing either unmodified accelerant or the modified accelerant, and the needle were attached to the Y-connector. The syringe holder and plunger cap were attached to the syringes. The syringes were subsequently simultaneous actuated.

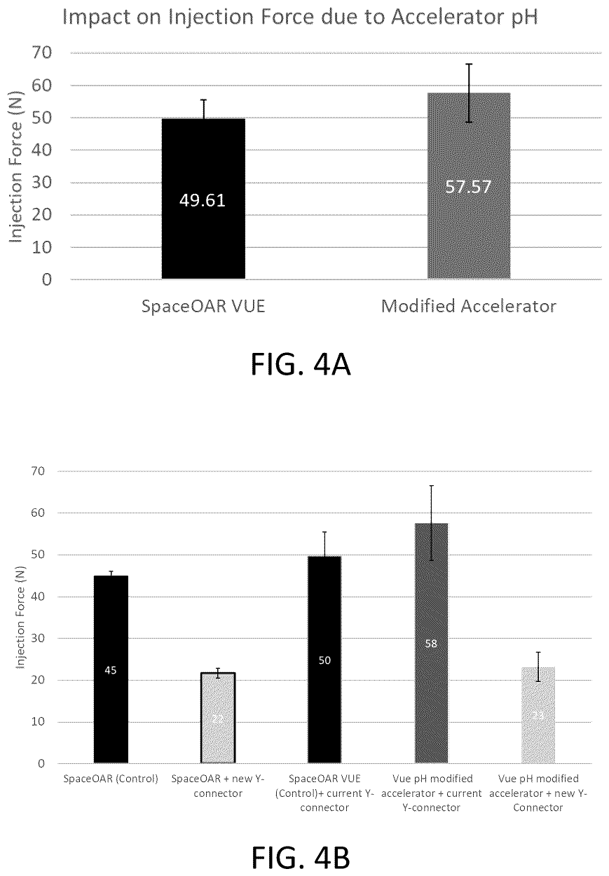

Maximum injection force was measured for injection with unmodified accelerator (measured pH=10.48) and for modified accelerator (measured pH=10.98). Both groups had n=15 samples. The data shown were generated by placing the device in an Instron test machine and depressing the plunger at a controlled rate. The maximum injection force was recorded for each sample. The results are shown in FIG. 4A, where error bars represent standard deviation. With reference to FIG. 3, the first branch lumen 118al and the second branch lumen 118bl of the commercial kit Y-connector were formed using hypotubes of 20 gauge (having an inside diameter of 0.603 mm and a cross-sectional area of 0.285 mm²).

To compensate for the increase in injection force that was unexpectedly exhibited for the modified accelerant, a modified Y-connector in which the first branch lumen 118al were formed using hypotubes having a larger inside diameter, typically, anywhere from 12 gauge (2.159 mm inside diameter) to 13 gauge (1.803 mm inside diameter) to 14 gauge (1.600 mm inside diameter) to 15 gauge (1.372 mm inside diameter) to 16 gauge (1.194 mm inside diameter) to 17 gauge (1.067 mm inside diameter) to 18 gauge (0.838 mm inside diameter) to 19 gauge (0.686 mm inside diameter), specifically, a 12 gauge hypotube (having an inside diameter of 2.159 mm and a cross-sectional area of 3.66 mm²). The results of the modification are illustrated in FIG. 4B, which shows a significant reduction in injection force. Also shown as injection force data generated with commercially available SpaceOAR® kits both with the commercial kit Y-connector and the modified Y-connector.

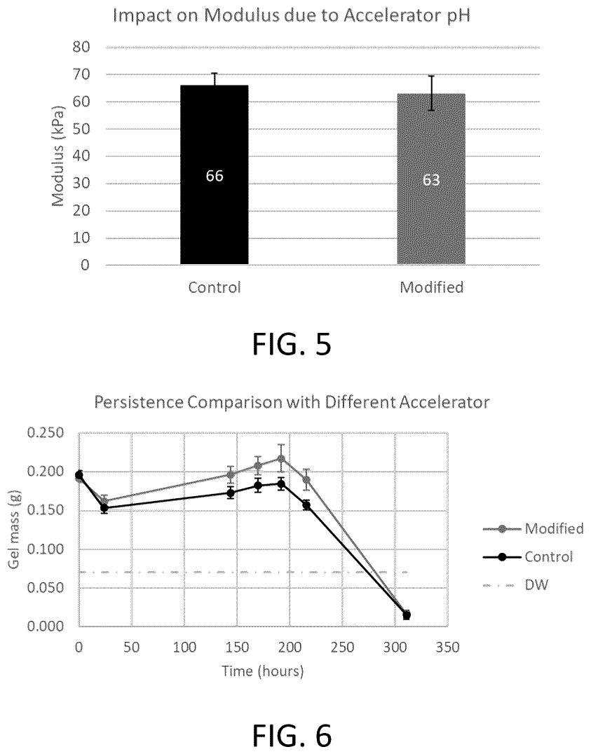

Gel plug modulus was measured for hydrogels formed with unmodified accelerator (measured pH=10.51) and for modified accelerator (measured pH=11.00). Both groups had n=15 samples. Gel plugs were cylindrical gel plugs 6 mm in diameter and 6 mm high. Modulus was measured using a uniaxial compression test on an Instron machine. A preload of approximately 0.01 N was applied to the gel plug. End of the test was a 2.75-millimeter (mm) extension or 40 newton (N) load. The modulus of the gel plug was taken from linear region of a force-displacement curve. The results are illustrated in FIG. 5, where error bars represent standard deviation. The results show that the impact on modulus between hydrogels formed using unmodified accelerator and hydrogels formed using modified accelerator were insignificant.

Gel persistence was measured for hydrogels formed with unmodified accelerator (measured pH=10.51) and for modified accelerator (measured pH=11.00). The data shown were generated by creating cylindrical gel plugs of 6 mm diameter and 6 mm height. Gel plugs were massed to record and initial weight. The gel plugs were then placed in a cell strainer with 40 um mesh. The filter was also weighed to obtain an initial mass. The gel plug was immersed in phosphate buffered saline (pH of 7.4) until the gel plug had undergone complete hydrolysis. The modified group had n=15 samples, and the unmodified group had n=7 samples. The results are shown in FIG. 6 where error bars represent standard deviation. Average gel mass as a function of time is shown for both groups. Average disappearance weight, which is the mass at which the sample is considered completely hydrolyzed, is represented by the horizontal line. The results show that the impact on gel persistence for hydrogels formed using unmodified accelerator and hydrogels formed using modified accelerator were insignificant.

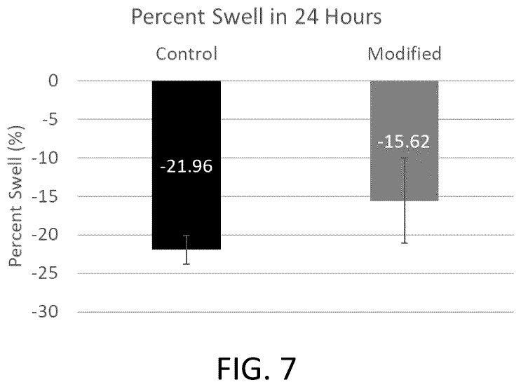

Percent swell was measured for hydrogels formed with unmodified accelerator (measured pH=10.51) and for modified accelerator (measured pH=11.00). The data shown were generated by creating cylindrical gel plugs of 6 mm diameter and 6 mm height. Gel plugs were massed initially to weight and then immersed in phosphate buffered saline (PBS) for 24 hours at 54 C. Mass was then recorded. % swell was calculated as the (day 1 mass-initial)/initial*100. The modified group had n=15 samples, and the unmodified group had n=7 samples. The results are shown in FIG. 7 where error bars represent standard deviation. Average percent swell after 24 hours is shown for both groups. The results show that the impact on percent swell for hydrogels formed using unmodified accelerator and hydrogels formed using modified accelerator were significant as determined by a paired t-test, p value 0.05.

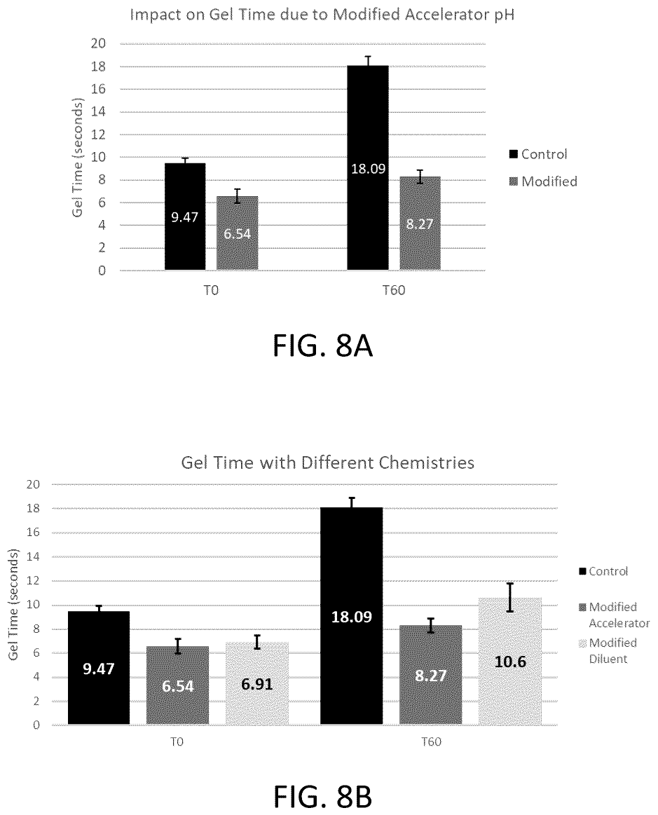

Gel time was measured for hydrogels formed with unmodified accelerator (measured pH=10.52) and for modified accelerator (measured pH=11.02). The data shown were generated by measuring time to viscosity change on a bench top test. A mini stir bar was used to mix 100 uL of accelerant and 99 uL of the precursor solution (combination of PEG powder and diluent). Gel time was defined as time taken for the material to adhere around the stir bar. T0 is +/−5 minutes of reconstitution of the PEG powder in the diluent. T60 is +/−60 minutes of reconstitution of the PEG powder and the diluent. Each group had n=15 samples, and all 15 samples were run in triplicate at T0 and T60. Average gel time is shown in FIG. 8A where error bars represent standard deviation. The results show that the gel time for hydrogels formed using modified accelerator were significantly less than the gel time for hydrogels formed using unmodified accelerator, both at T0 and T60.

### Example 2

To determine the effect of modifying buffered diluent solution pH on gel properties, five tests were completed, specifically, gel persistence, percent swell, gel modulus, maximum injection force, and gel time.

To modify buffered diluent solution, a stir bar was placed into a 100 mL beaker, and 15 diluent syringes from existing SpaceOAR® Vue kits were expelled into the 100 mL beaker. The buffered diluent solution contained a combination of trilysine and dibasic sodium phosphate as buffering agents. Once all diluent syringes from the kits were emptied into the beaker, the initial pH of the combined solution was measured. NaOH was then added to the combined accelerant solution to increase the pH of the solution. In particular, 6.0N NaOH was titrated until the pH was about 0.6 pH points higher than the initial pH. Approximately 300 μL of NaOH was added. When the desired pH was reached, the diluent syringes were refilled with the modified buffered diluent solution by pulling 5 mL of the solution from the 100 mL beaker back into the syringes. The existing kits also included a vial containing 8-arm PEG powder in which approximately 60% of the polymer arms have succinimidyl glutarate end groups and approximately 40% of the polymer arms have 2,3,5-triiiodobenzamide end groups, an accelerant syringe containing a buffered accelerant solution containing sodium tetraborate decahydrate and dibasic sodium phosphate as buffering agents (pH=10.5), a Y-connector, an 18 G×15 cm needle, a syringe holder, a plunger cap and a vial adapter.

Using the vial adaptor, either modified or unmodified buffered diluent solution was introduced into the vial to dissolve the PEG powder and form a modified or unmodified precursor solution, respectively, which was drawn back into the syringe. The syringe containing the modified or unmodified precursor solution, the syringe containing the accelerant, and the needle were then attached to the Y-connector. The syringe holder and plunger cap were attached to the syringes, after which the syringes were simultaneous actuated.

Gel time was measured for hydrogels formed with unmodified buffered diluent solution (measured pH=4.2) and for modified buffered diluent solution (measured pH=4.83). Each group had n=15 samples, and all 15 samples were run in triplicate at T0 and T60. Average gel time is shown in FIG. 8B where error bars represent standard deviation. The results show that the gel time for hydrogels formed using modified buffered diluent solution is significantly less than the gel time for hydrogels formed using unmodified buffered diluent solution, both at T0 and T90. Also included in FIG. 8B are the modified and unmodified accelerant data from FIG. 8A.

1. A system for forming a hydrogel composition that comprises a first reservoir containing an iodinated polymer composition that comprise an iodinated polymer having multiple cyclic imide ester groups, a second reservoir containing a buffered diluent solution having a pH ranging from 3.8-4.2 that comprises water, a first buffering agent and a polyamine compound, and a third reservoir containing a buffered accelerant solution having a pH ranging from 10.8-11.2 that comprises water and a second buffering agent.

2. The system of claim 1, wherein upon dissolving the iodinated polymer composition in the buffered diluent solution to form a buffered precursor solution containing water, the first buffering agent, the iodinated polymer and the polyamine, and upon mixing the buffered precursor solution with the buffered accelerant, crosslinking between the iodinated polymer and the polyamine leads to hydrogel formation.

3. The system of claim 2, wherein a gel formation time for hydrogel formation ranges from 5 to 8 seconds when the buffered precursor solution is combined with the buffered accelerant solution within 10 minutes of forming the precursor solution.

4. The system of claim 1, wherein the first buffering agent comprises a phosphate buffering agent.

5. The system of claim 1, wherein the second buffering agent comprises a borate buffering agent and a phosphate buffering agent.

6. The system of claim 1, wherein the first reservoir is a vial, wherein the second reservoir is a first syringe comprising a first syringe barrel terminating in a connector and a first plunger, and wherein the third reservoir is a second syringe comprising a second syringe barrel terminating in a connector and a second plunger.

7. The system of claim 6, further comprising (a) a needle or a tube and (b) a Y-connector that comprises a first branch lumen having a first end and a second end, a second branch lumen having a first end and a second end, and a common lumen having a first end and a second end, the first end of the first branch lumen and the first end of the second branch lumen in fluid communication with the first end of the common lumen, the second end of the first branch lumen terminating at a connector which is configured to connect with the connector of the first syringe barrel, the second end of the second branch lumen terminating at a connector which is configured to connect with the connector of the second syringe barrel, and the second end of the common lumen terminating at a connector configured to connect to a connector of the needle or the tube.

8. The system of claim 7, wherein the first branch lumen and the second branch lumen have an internal diameter ranging from 12 gauge to 19 gauge.

9. The system of claim 7, wherein the system further comprises one, two of all three of the following: a syringe holder that is configured to hold the first and second syringe barrels in a fixed relationship, a plunger cap that is configured to hold the first and second plungers in a fixed relationship, and a vial adapter configured for providing fluid communication between the syringe barrel and the vial.

10. A kit comprising a sterile package comprising components of the system of claim 7 in one or more trays.

11. A method of using the kit of claim 10, comprising:
injecting the buffered diluent solution from the first syringe into the vial containing the iodinated polymer composition, thereby forming a buffered precursor solution containing water, the first buffering agent, the iodinated polymer and the polyamine;
pulling the buffered precursor solution back into the first syringe;
connecting the first syringe barrel of the first syringe to the first branch lumen of the Y-connector;
connecting the second syringe barrel of the second syringe to the second branch lumen of the Y-connector;
connecting the needle or tube to the common lumen of the Y-connector, and
simultaneously pressing the first plunger and the second plunger into the first syringe barrel and the second syringe barrel, respectively.

12. A system for forming a hydrogel composition that comprises a vial containing an iodinated polymer composition that comprise an iodinated polymer having multiple cyclic imide ester groups, a first syringe comprising a first syringe barrel terminating in a connector and a first plunger, the first syringe containing a buffered diluent solution having a pH ranging from 3.8-4.2 that comprises water, a first buffering agent and a polyamine compound, and a second syringe comprising a second syringe barrel terminating in a connector and a second plunger, the second syringe containing a buffered accelerant solution having a pH ranging from 10.8-11.2 that comprises water and a second buffering agent, wherein upon dissolving the iodinated polymer composition in the buffered diluent solution to form a buffered precursor solution containing water, the first buffering agent, the iodinated polymer and the polyamine, and upon mixing the buffered precursor solution with the buffered accelerant, crosslinking between the iodinated polymer and the polyamine leads to hydrogel formation, wherein a gel formation time for the hydrogel formation ranges from 5 to 8 seconds when the buffered precursor solution is combined with the buffered accelerant solution within 10 minutes of forming the precursor solution.

13. A system for forming a hydrogel composition that comprises a first reservoir containing an iodinated polymer composition that comprise an iodinated polymer having multiple cyclic imide ester groups, a second reservoir containing a buffered diluent solution having a pH ranging from 4.4-5.5 that comprises water, a first buffering agent and a polyamine compound, and a third reservoir containing a buffered accelerant solution having a pH ranging from 10.2-10.6 that comprises water and a second buffering agent.

14. The system of claim 13, wherein upon dissolving the iodinated polymer composition in the buffered diluent solution to form a buffered precursor solution containing water, the first buffering agent, the iodinated polymer and the polyamine, and upon mixing the buffered precursor solution with the buffered accelerant, crosslinking between the iodinated polymer and the polyamine leads to hydrogel formation.

15. The system of claim 14, wherein a gel formation time for hydrogel formation ranges from 5 to 8 seconds when the buffered precursor solution is combined with the buffered accelerant solution within 10 minutes of forming the precursor solution.

16. The system of claim 13, wherein the first buffering agent comprises a phosphate buffering agent.

17. The system of claim 13, wherein the second buffering agent comprises a borate buffering agent and a phosphate buffering agent.

18. The system of claim 13, wherein the first reservoir is a vial, wherein the second reservoir is a first syringe comprising a first syringe barrel terminating in a connector and a first plunger, and wherein the third reservoir is a second syringe comprising a second syringe barrel terminating in a connector and a second plunger.

19. The system of claim 18, further comprising (a) a needle or a tube and (b) a Y-connector that comprises a first branch lumen having a first end and a second end, a second branch lumen having a first end and a second end, and a common lumen having a first end and a second end, the first end of the first branch lumen and the first end of the second branch lumen in fluid communication with the first end of the common lumen, the second end of the first branch lumen terminating at a connector which is configured to connect with the connector of the first syringe barrel, the second end of the second branch lumen terminating at a connector which is configured to connect with the connector of the second syringe barrel, and the second end of the common lumen terminating at a connector configured to connect to a connector of the needle or the tube.

20. The system of claim 19, wherein the first branch lumen and the second branch lumen have an internal diameter ranging from 12 gauge to 19 gauge.
